(19) United States
(12) Patent Application Publication
(10) Pub. No.: US 2013/0164351 A1
(43) Pub. Date: Jun. 27, 2013

(54) METHODS OF TREATING BACTERIAL INFECTIONS THROUGH PULMONARY DELIVERY OF FUSIDIC ACID

(71) Applicant: Prabhavathi Fernandes, Chapel Hill, NC (US)

(72) Inventor: Prabhavathi Fernandes, Chapel Hill, NC (US)

(73) Assignee: CEMPRA PHARMACEUTICALS INC., Chapel Hill, NC (US)

(21) Appl. No.: 13/819,917

(22) PCT Filed: Aug. 15, 2011
(86) PCT No.: PCT/US2011/047771

§ 371 Date: Feb. 28, 2013

Related U.S. Application Data
(60) Provisional application No. 61/378,032, filed on Aug. 30, 2010.

(51) Int. Cl. A61K 9/00; A61K 45/06; A61K 31/575

(52) U.S. Cl. CPC A61K 9/007; A61K 31/575; A61K 45/06
USPC 424/400; 514/182; 514/171

(57) ABSTRACT

Methods for the treatment of bacterial infections in the respiratory system of a subject, such as the lungs of a subject, using fusidic acid alone or in combination with a second bacterial agent such as tobramycin, amikacin, fosfomycin or levofloxacin are described.

### BACKGROUND OF THE INVENTION

Fusidic acid (FA) is a tetracyclic triterpenoid or fusidane (steroidal) antibiotic derived from the fungus Fusidium coccineum that inhibits bacterial protein synthesis. FA is effective against gram-positive bacteria such as Staphylococcus species and Corynebacterium species (L. Verbist, J. Antimicro. Chemo. 25, Suppl. B, 1-5 (1990); A. Bryskier, Fusidic Acid, Chapter 23, in Antimicrobial Agents Antibacterials and Antifungals (Andre Bryskier, Ed., ASM Press, Washington, USA, 2005)). FA also has moderate activity against Group A beta-hemolytic streptococci, including Streptococcus pyogenes (L. Verbist, J. Antimicro. Chemo. 25, Suppl. B, 1-5 (1990); A. Bryskier, Fusidic Acid, Chapter 23, in Antimicrobial Agents: Antibacterials and Antifungals (Andre Bryskier, Ed., ASM Press, Washington, USA, 2005); Skov et al., Diag. Micro. Infect. Dis. 40:111-116 (2001)).

FA was developed for clinical use in the 1960s and it is approved for human use outside of the United States, such as in the UK, Canada, Europe, Israel, Australia and New Zealand. It is typically prescribed at doses of 500 mg TID for treating skin and skin structure infections caused by Staphylococcus aureus (A. Bryskier, Fusidic Acid, Chapter 23, in Antimicrobial Agents Antibacterials and Antifungals (Andre Bryskier, Ed., ASM Press, Washington, USA, 2005); Collignon et al., Int'l J. Antimicrobial Agents 12:S45-S58 (1999); D. Spelman, Int'l J. Antimicrobial Agents 12:S59-S66 (1999)), although some physicians have routinely prescribed the compound at 500 mg BID for treating skin and skin structure infections due to the long half-life of the compound (Fusidic Acid, in Principles and Practice of Infectious Diseases, 6^{th} ed. (Mandell et al. eds., Elsevier, 2006)).

Treatment using FA has been well studied and it is generally regarded as safe when administered to humans, as evidenced by the fact that the drug has been in continuous use since 1968 in various parts of the world. There are, however, several characteristics of FA that have suggested against the use of the drug against a wider spectrum of bacteria and in the treatment in additional types of infection. For example, approved dosing regimens have been shown to select for bacterial resistance, such as in S. aureus. Approved dosing regimens provide low multiples of the MIC and as a result, S. aureus resistant mutants can be selected after the first day of dosing. Once resistance has developed, FA is not effective against the resistant strains. Resistance is reported to occur if FA is used as a single drug as the resistance frequency at 4 and 8 times the MIC is in the range of 10⁻⁶ or 10⁻⁸ (Evans et al., J. Clin. Path. 19:555-560 (1966); Hansson et al., J. Mol. Biol. 348:939-949 (2005), Jensen et al., Acta Pathol Microbiol Scand. 60:271-284 (1964); Besier et al., Antimicrob. Agents Chemo., 49(4):1426-1431 (2005); Gemmell et al., J. Antimicrobial Chemo. 57:589-608 (2006); Howden et al., Clin. Infect. Disease 42:394-400 (2006)).

The dosage of the drug cannot be simply increased as a means of avoiding development of resistance. It is difficult to achieve high concentrations of free (unbound) FA in the blood due to the substantial protein binding of the drug (approximately 95-97%) (K. Christiansen, International Journal of Antimicrobial Agents 12:S3-S9 (1999); Coutant et al., Diagn Microbiol Infect Dis 25:9-13 (1996); D. Reeves, J. Antimicrob. Chemo. 20:467-476 (1987); J. Turnidge, Int'l J. Antimicrobial Agents 12:S23-S34 (1999); Rieutord et al., Int'l J. Pharmaceutics 119:57-64 (1995)). Moreover, high dosages of FA are not well-tolerated by patients receiving the drug. High doses of FA (e.g., 1 gram TID) are required if the drug is to be used in the treatment of bone and joint infections, less susceptible bacteria and other serious infections. However, treatment regimens using high doses of the drug induce nausea and vomiting and are rejected by patients (Fusidic Acid, in Principles and Practice of Infectious Diseases, 6^{th} ed. (Mandell et al. eds., Elsevier, 2006); K. Christiansen, International Journal of Antimicrobial Agents 12:S3-S9 (1999); Nordin et al., Eur. J. Clin. Res. 5:97-106 (1994)).

In view of the tremendous costs associated with the de novo development of new anti-bacterials, expanding the indications for drugs that have already been demonstrated to be safe and effective is strongly needed. Finding new uses and means for administering FA would broaden the population of bacterial infections against which FA could be used and thus meet this need.

### BRIEF SUMMARY OF THE INVENTION

The present invention generally provides methods of treating bacterial infections in the respiratory system of a subject, such as the lungs of a subject, using fusidic acid alone or in combination with a second bacterial agent such as tobramycin, amikacin, fosfomycin or levofloxacin. Such subjects may have an underlying disease or condition that makes them more susceptible to bacterial infections of the respiratory system, such as cystic fibrosis.

Thus, in a first embodiment the present invention provides methods of treating a bacterial infection in the respiratory system of a subject, including a subject having cystic fibrosis, comprising administering via inhalation a therapeutically effective amount of a pharmaceutical composition comprising fusidic acid, or a pharmaceutically acceptable salt thereof, to the respiratory system of a subject having a bacterial infection therein.

In this embodiment the bacterial infection is an infection caused by one or more bacterial species selected from the group consisting of Staphylococcus aureus (methicillin-resistant or -susceptible), Pseudomonas aeruginosa, Bacillus anthracis, and Burkholderia cepacia. In certain aspects of this embodiment the bacterial infection is a chronic bacterial infection.

The therapeutically effective amount of the pharmaceutical composition is an amount sufficient to treat the bacterial infection in the subject. In one aspect, the therapeutically effective amount comprises between about 200 mg and about 1500 mg fusidic acid, or a pharmaceutically acceptable salt thereof. In another aspect, the therapeutically effective amount comprises between about 400 mg and about 800 mg fusidic acid, or a pharmaceutically acceptable salt thereof.

In varying aspects, the pharmaceutical composition is administered to the subject once, twice or thrice daily.

In a second embodiment the present invention provides methods for delivering fusidic acid to a subject, including a subject having cystic fibrosis, comprising administering via inhalation a therapeutically effective amount of a pharmaceutical composition comprising fusidic acid, or a pharmaceutically acceptable salt thereof, to the respiratory system of a subject.

In certain aspects of this embodiment, the respiratory system of the subject has a bacterial infection, wherein the infection is caused by one or more bacterial species selected from the group consisting of Staphylococcus aureus (methicillin-resistant or -susceptible), Pseudomonas aeruginosa, Bacillus anthracis, and Burkholderia cepacia. The bacterial infection can be a chronic bacterial infection.

The therapeutically effective amount of the pharmaceutical composition is an amount sufficient to treat the bacterial infection in the subject. In one aspect, the therapeutically effective amount comprises between about 200 mg and about 1500 mg fusidic acid, or a pharmaceutically acceptable salt thereof. In another aspect, the therapeutically effective amount comprises between about 400 mg and about 800 mg fusidic acid, or a pharmaceutically acceptable salt thereof.

In one aspect of this embodiment, the method is practiced about every 8 hours. In another aspect of this embodiment, the method is practiced about every 12 hours. In a further aspect of this embodiment, the method is practiced about every 24 hours.

In a third embodiment the present invention provides methods of treating a bacterial infection in the respiratory system of a subject, including a subject having cystic fibrosis, comprising administering via inhalation a dose of about 4.0 ml or less of a nebulized aerosol formulation comprising from about 200 mg to about 1500 mg of fusidic acid, or a pharmaceutically acceptable salt thereof, in a time period of about 10 minutes or less to a subject having a bacterial infection of the respiratory system.

In one aspect, this method is practiced using an inhalation device having a rate of aerosol output of not less than about 4 ul/sec, that releases about 75% of the loaded dose, and that produces aerosol particles having particle sizes between about 1 micron and about 5 micron. In a particular aspect, the dose is about 3.75 ml or less of the nebulized aerosol formulation, or the dose is about 3.75 ml of the nebulized aerosol formulation. In other aspects, the nebulized aerosol formulation comprises from about 400 mg to about 800 mg of fusidic acid, or a pharmaceutically acceptable salt thereof, or it comprises from about 300 mg to about 600 mg of fusidic acid, or a pharmaceutically acceptable salt thereof. In further aspects, the nebulized aerosol formulation is administered in a time period of about 8 minutes or less, or about 6 minutes or less.

In this embodiment the bacterial infection is an infection caused by one or more bacterial species selected from the group consisting of Staphylococcus aureus (methicillin-resistant or -susceptible), Pseudomonas aeruginosa, Bacillus anthracis, and Burkholderia cepacia. In certain aspects of this embodiment the bacterial infection is a chronic bacterial infection.

In each embodiment of the invention the subject can be a human.

In certain aspects, the first, second and third embodiments of the invention further comprise administering a therapeutically effective amount of a pharmaceutical composition comprising tobramycin, or a pharmaceutically acceptable salt thereof, to the subject. The pharmaceutical composition comprising tobramycin can be administered to the subject prior to, concurrently with, or after administering the fusidic acid, or pharmaceutically acceptable salt thereof, to the subject. The pharmaceutical composition comprising tobramycin is administered to the respiratory system of the subject by inhalation, or it is administered to the subject intravenously or intramuscularly.

In certain aspects, the first, second and third embodiments of the invention further comprise administering a therapeutically effective amount of a pharmaceutical composition comprising amikacin, or a pharmaceutically acceptable salt thereof, to the subject. The pharmaceutical composition comprising amikacin can be administered to the subject prior to, concurrently with, or after administering the fusidic acid, or pharmaceutically acceptable salt thereof, to the subject. The pharmaceutical composition comprising amikacin is administered to the respiratory system of the subject by inhalation, or it is administered to the subject intravenously or intramuscularly.

In certain aspects, the first, second and third embodiments of the invention further comprise administering a therapeutically effective amount of a pharmaceutical composition comprising fosfomycin, or a pharmaceutically acceptable salt thereof, to the subject. The pharmaceutical composition comprising fosfomycin can be administered to the subject prior to, concurrently with, or after administering the fusidic acid, or pharmaceutically acceptable salt thereof, to the subject. The pharmaceutical composition comprising fosfomycin is administered to the respiratory system of the subject by inhalation, or it is administered to the subject orally.

In certain aspects, the first, second and third embodiments of the invention further comprise administering a therapeutically effective amount of a pharmaceutical composition comprising levofloxacin, or a pharmaceutically acceptable salt thereof, to the subject. The pharmaceutical composition comprising levofloxacin can be administered to the subject prior to, concurrently with, or after administering the fusidic acid, or pharmaceutically acceptable salt thereof, to the subject. The pharmaceutical composition comprising levofloxacin is administered to the respiratory system of the subject by inhalation, or it is administered to the subject orally or by intravenous infusion.

In certain aspects, the first, second and third embodiments of the invention further comprise administering a bronchodilator to said subject in an amount sufficient to inhibit bronchoconstriction. The bronchodilator can be administered to the subject prior to or concurrently with any of the pharmaceutical compositions or nebulized aerosol formulations comprising an antibacterial agent of the present invention.

In certain aspects, the treatment provided in the first or third embodiment of the invention is a bactericidal treatment. In other aspects the treatment is bacteriostatic.

In certain aspects, the methods provided in the second embodiment of the invention result in bactericidal treatment of a bacterial infection in the respiratory system of the subject. In other aspects the treatment methods result in bacteriostatic treatment of a bacterial infection in the respiratory system of the subject.

In a fourth embodiment, the present invention provides use of fusidic acid or a pharmaceutically acceptable salt thereof in the preparation of a pharmaceutical composition for treating via inhalation a bacterial infection in the respiratory system of a subject.

In a fifth embodiment, the present invention provides use of fusidic acid or a pharmaceutically acceptable salt thereof in the preparation of a pharmaceutical composition for treating via inhalation a bacterial infection in the respiratory system of a subject having cystic fibrosis.

In certain aspects of the fourth and fifth embodiments, the bacterial infection is an infection caused by one or more bacterial species selected from the group consisting of Staphylococcus aureus (methicillin-resistant or -susceptible), Pseudomonas aeruginosa, Bacillus anthracis, and Burkholderia cepacia.

In certain aspects of the fourth and fifth embodiments, the pharmaceutical composition comprises between about 200 mg and about 1500 mg of fusidic acid, or a pharmaceutically acceptable salt thereof.

In certain aspects of the fourth and fifth embodiments, the pharmaceutical composition comprises between about 400 mg and about 800 mg of fusidic acid, or a pharmaceutically acceptable salt thereof.

In certain aspects of the fourth and fifth embodiments, the pharmaceutical composition is administered to the subject once, twice or thrice daily.

In a sixth embodiment, the present invention provides use of fusidic acid or a pharmaceutically acceptable salt thereof in the preparation of a pharmaceutical composition for treating via inhalation a bacterial infection in the respiratory system of a subject, wherein the pharmaceutical composition comprises a dose of about 4.0 ml or less of a nebulized aerosol formulation comprising from about 200 mg to about 1500 mg of fusidic acid, or a pharmaceutically acceptable salt thereof, for delivery in a time period of about 10 minutes or less.

In a seventh embodiment, the present invention provides use of fusidic acid or a pharmaceutically acceptable salt thereof in the preparation of a pharmaceutical composition for treating via inhalation a bacterial infection in the respiratory system of a subject having cystic fibrosis, wherein the pharmaceutical composition comprises a dose of about 4.0 ml or less of a nebulized aerosol formulation comprising from about 200 mg to about 1500 mg of fusidic acid, or a pharmaceutically acceptable salt thereof, for delivery in a time period of about 10 minutes or less.

In certain aspects of the sixth and seventh embodiments, the dose is administered via an inhalation device having a rate of aerosol output of not less than about 4 ul/sec, that releases about 75% of the loaded ose, and that produces aerosol particles having particle sizes between about 1 micron and about 5 micron.

In certain aspects of the sixth and seventh embodiments, the dose is about 3.75 ml or less of the nebulized aerosol formulation.

In certain aspects of the sixth and seventh embodiments, the nebulized aerosol formulation comprises from about 400 mg to about 800 mg of fusidic acid, or a pharmaceutically acceptable salt thereof.

In certain aspects of the sixth and seventh embodiments, the nebulized aerosol formulation comprises from about 300 mg to about 600 mg of fusidic acid, or a pharmaceutically acceptable salt thereof.

In certain aspects of the sixth and seventh embodiments, the bacterial infection is an infection caused by one or more bacterial species selected from the group consisting of Staphylococcus aureus (methicillin-resistant or -susceptible), Pseudomonas aeruginosa, Bacillus anthracis, and Burkholderia cepacia.

In certain aspects of the fourth, fifth, sixth and seventh embodiments, the bacterial infection is a chronic bacterial infection.

In certain aspects of the fourth, fifth, sixth and seventh embodiments, the subject is a human.

In certain aspects of the fourth, fifth, sixth and seventh embodiments, the pharmaceutical composition further comprises tobramycin or a pharmaceutically acceptable salt thereof.

In certain aspects of the fourth, fifth, sixth and seventh embodiments, the pharmaceutical composition further comprises amikacin or a pharmaceutically acceptable salt thereof.

In certain aspects of the fourth, fifth, sixth and seventh embodiments, the pharmaceutical composition further comprises fosfomycin or a pharmaceutically acceptable salt thereof.

In certain aspects of the fourth, fifth, sixth and seventh embodiments, the pharmaceutical composition further comprises levofloxacin or a pharmaceutically acceptable salt thereof.

In certain aspects of the fourth, fifth, sixth and seventh embodiments, the pharmaceutical composition further comprises a bronchodilator in an amount sufficient to inhibit bronchoconstriction.

In certain aspects of the sixth and seventh embodiments, the dose is a bactericidal dose or a bacteriostatic dose.

The present invention is also provides a kit comprising one or more of the pharmaceutical compositions described herein and a means for administering the compositions to a subject.

### DESCRIPTION OF THE DRAWINGS

FIG. 1: Synergy OF CEM-102 (fusidic acid)+tobramycin against one B. cepacia strain.

### DETAILED DESCRIPTION OF THE INVENTION

Through studies and the diligent efforts of the inventors, and as disclosed herein, it has been discovered that bacterial infections of the respiratory system, such as the lungs, caused by bacterial species such as Staphylococcus aureus can be successfully treated using fusidic acid when this antibacterial agent is administered to the respiratory system of a subject. It has further been found that the use of fusidic acid, in combination with a second antibacterial agent such as tobramycin, amikacin, fosfomycin or levofloxacin, represents an improvement over available means for treating bacterial infections of the respiratory system caused by organisms such as Pseudomonas aeruginosa and Burkholderia cepacia. As such, the present invention provides methods for the treatment of bacterial infections in the respiratory system of a subject using fusidic acid, either alone or in combination with one or more additional antibacterial agent. The methods of the present invention can be practiced by administering to the respiratory system of a subject a pharmaceutical composition comprising fusidic acid, alone or in combination with an additional antibacterial agent as disclosed herein.

The present invention thus provides methods of treating a bacterial infection in the respiratory system of a subject, comprising administering via inhalation a therapeutically effective amount of a pharmaceutical composition comprising fusidic acid, or a pharmaceutically acceptable salt thereof, to the respiratory system of a subject having a bacterial infection therein. In certain aspects, one or more additional antibacterial agents are administered to the subject. In one of these aspects, the present invention provides methods of treating a bacterial infection in the respiratory system of a subject, comprising (i) administering via inhalation a therapeutically effective amount of a pharmaceutical composition comprising fusidic acid, or a pharmaceutically acceptable salt thereof, to the respiratory system of a subject having a bacterial infection therein, and (ii) administering a therapeutically effective amount of a pharmaceutical composition comprising tobramycin, or a pharmaceutically acceptable salt thereof, to the subject. In another of these aspects, the present invention provides methods of treating a bacterial infection in the respiratory system of a subject, comprising (i) administering via inhalation a therapeutically effective amount of a pharmaceutical composition comprising fusidic acid, or a pharmaceutically acceptable salt thereof, to the respiratory system of a subject having a bacterial infection therein, and (ii) administering a therapeutically effective amount of a pharmaceutical composition comprising amikacin, or a pharmaceutically acceptable salt thereof, to the subject. In yet another of these aspects, the present invention provides methods of treating a bacterial infection in the respiratory system of a subject, comprising (i) administering via inhalation a therapeutically effective amount of a pharmaceutical composition comprising fusidic acid, or a pharmaceutically acceptable salt thereof, to the respiratory system of a subject having a bacterial infection therein, and (ii) administering a therapeutically effective amount of a pharmaceutical composition comprising fosfomycin, or a pharmaceutically acceptable salt thereof, to the subject. In still another of these aspects, the present invention provides methods of treating a bacterial infection in the respiratory system of a subject, comprising (i) administering via inhalation a therapeutically effective amount of a pharmaceutical composition comprising fusidic acid, or a pharmaceutically acceptable salt thereof, to the respiratory system of a subject having a bacterial infection therein, and (ii) administering a therapeutically effective amount of a pharmaceutical composition comprising levofloxacin, or a pharmaceutically acceptable salt thereof, to the subject.

In a related embodiment, the present invention provides methods for delivering fusidic acid to a subject, comprising administering via inhalation a therapeutically effective amount of a pharmaceutical composition comprising fusidic acid, or a pharmaceutically acceptable salt thereof, to the respiratory system of a subject. In certain aspects of this embodiment, the respiratory system of the subject has a bacterial infection. In other aspects, one or more additional antibacterial agents are administered to the subject. In one of these other aspects, the present invention provides methods for delivering fusidic acid to a subject, comprising (i) administering via inhalation a therapeutically effective amount of a pharmaceutical composition comprising fusidic acid, or a pharmaceutically acceptable salt thereof, to the respiratory system of a subject, and (ii) administering a therapeutically effective amount of a pharmaceutical composition comprising tobramycin, or a pharmaceutically acceptable salt thereof, to the subject. In another of these other aspects, the present invention provides methods for delivering fusidic acid to a subject, comprising (i) administering via inhalation a therapeutically effective amount of a pharmaceutical composition comprising fusidic acid, or a pharmaceutically acceptable salt thereof, to the respiratory system of a subject, and (ii) administering a therapeutically effective amount of a pharmaceutical composition comprising amikacin, or a pharmaceutically acceptable salt thereof, to the subject. In yet another of these other aspects, the present invention provides methods for delivering fusidic acid to a subject, comprising (i) administering via inhalation a therapeutically effective amount of a pharmaceutical composition comprising fusidic acid, or a pharmaceutically acceptable salt thereof, to the respiratory system of a subject, and (ii) administering a therapeutically effective amount of a pharmaceutical composition comprising fosfomycin, or a pharmaceutically acceptable salt thereof, to the subject. In still another of these other aspects, the present invention provides methods for delivering fusidic acid to a subject, comprising (i) administering via inhalation a therapeutically effective amount of a pharmaceutical composition comprising fusidic acid, or a pharmaceutically acceptable salt thereof, to the respiratory system of a subject, and (ii) administering a therapeutically effective amount of a pharmaceutical composition comprising levofloxacin, or a pharmaceutically acceptable salt thereof, to the subject.

The present invention further provides methods of treating a bacterial infection in the respiratory system of a subject, comprising administering via inhalation a relatively small volume of a pharmaceutical composition comprising a therapeutically effective amount of fusidic acid, or a pharmaceutically acceptable salt thereof, over a relatively short period of time to a subject having a bacterial infection of the respiratory system. This aspect of the invention allows delivery of fusidic acid through aerosolization of a small volume of drug into aerosolized particles of between about 1 and about 5 microns in size, delivered by inhalers having a high output rate and high efficiency, thereby providing efficacious delivery of fusidic acid into areas of the respiratory system having a susceptible microbial infection, such as a Staphylococcus aureus infection. The aerosol formulations preferably contain minimal yet efficacious amounts of fusidic acid, formulated in the smallest practical volume of a physiologically acceptable solution, that are well-tolerated by subjects but that do not induce undesirable side effects such as bronchospasm and cough. Further, direct delivery of high concentrations of fusidic acid to the respiratory system by aerosolization result in maximization of sputum levels of drug and in minimization of serum levels of the drug. Thus, administration of fusidic acid by aerosolization has the advantage of reducing the potential for systemic toxicity while providing efficacious concentrations of fusidic acid in the sputum. The bronchial barrier restricts the movement of aerosolized fusidic acid and prevents it from reaching high systemic levels.

Thus, in accordance with one aspect of the present invention, methods are provided for treating a bacterial infection in the respiratory system of a subject, comprising administering via inhalation a dose of about 4.0 ml or less of a nebulized aerosol formulation comprising from about 200 to about 1500 mg of fusidic acid, or a pharmaceutically acceptable salt thereof, in a time period of about 10 minutes or less to a subject having a bacterial infection of the respiratory system. In certain aspects, one or more additional antibacterial agents are administered to the subject. In one of these aspects, the present invention provides methods for treating a bacterial infection in the respiratory system of a subject, comprising (i) administering via inhalation a dose of about 4.0 ml or less of a nebulized aerosol formulation comprising from about 200 to about 1500 mg of fusidic acid, or a pharmaceutically acceptable salt thereof, in a time period of about 10 minutes or less, to a subject having a bacterial infection of the respiratory system, and (ii) administering a therapeutically effective amount of a pharmaceutical composition comprising tobramycin, or a pharmaceutically acceptable salt thereof, to the subject. In another of these aspects, the present invention provides methods for treating a bacterial infection in the respiratory system of a subject, comprising (i) administering via inhalation a dose of about 4.0 ml or less of a nebulized aerosol formulation comprising from about 200 to about 1500 mg of fusidic acid, or a pharmaceutically acceptable salt thereof, in a time period of about 10 minutes or less, to a subject having a bacterial infection of the respiratory system, and (ii) administering a therapeutically effective amount of a pharmaceutical composition comprising amikacin, or a pharmaceutically acceptable salt thereof, to the subject. In yet another of these aspects, the present invention provides methods for treating a bacterial infection in the respiratory system of a subject, comprising (i) administering via inhalation a dose of about 4.0 ml or less of a nebulized aerosol formulation comprising from about 200 to about 1500 mg of fusidic acid, or a pharmaceutically acceptable salt thereof, in a time period of about 10 minutes or less, to a subject having a bacterial infection of the respiratory system, and (ii) administering a therapeutically effective amount of a pharmaceutical composition comprising fosfomycin, or a pharmaceutically acceptable salt thereof, to the subject. In still another of these aspects, the present invention provides methods for treating a bacterial infection in the respiratory system of a subject, comprising (i) administering via inhalation a dose of about 4.0 ml or less of a nebulized aerosol formulation comprising from about 200 to about 1500 mg of fusidic acid, or a pharmaceutically acceptable salt thereof, in a time period of about 10 minutes or less, to a subject having a bacterial infection of the respiratory system, and (ii) administering a therapeutically effective amount of a pharmaceutical composition comprising levofloxacin, or a pharmaceutically acceptable salt thereof, to the subject.

### Bacterial Infection

As used herein, the term bacterial infection refers to an infection caused by one or more bacterial species selected from the group consisting of staphylococci, including coagulase-negative staphylococci and coagulase-positive staphylococci, streptococci, including Group A beta hemolytic streptococci, non-Group A beta hemolytic streptococci and viridans group streptococci, enterococci, Nesseria species, Clostridium species, Bordetella species, Bacillus species and Corynebacterium species. In particular, the bacterial infection is an infection caused by one or more bacterial species selected from the group consisting of Staphylococcus aureus (methicillin-resistant and -susceptible), Staphylococcus epidermidis, Staphylococcus hemolyticus, Staphylococcus saprophyticus, Staphylococcus lugdunensis, Staphylococcus capitis, Staphylococcus caprae, Staphylococcus saccharolyticus, Staphylococcus simulans, Staphylococcus warneri, Staphylococcus hominis, Staphylococcus intermedius, Staphylococcus pseudointermedius, Staphylococcus lyricus, Streptococcus pyogenes, Streptococcus agalactiae, Streptococcus dysgalactiae subspecies dysgalactiae, Streptococcus anginosus, Streptococcus mitis, Streptococcus salivarius, Streptococcus bovis, Streptococcus mutans, Pseudomonas aeruginosa, Neisseria gonorrhoeae, Neisseria meningitidis, Bacillus anthracis, Bordetella pertussis, Burkholderia cepacia, Clostridium difficile, Enterococcus faecalis, Enterococcus faecium and Corynebacterium diphtheriae. In particular aspects, the bacterial infection is an infection caused by one or more of Staphylococcus aureus (methicillin-resistant or -susceptible), Pseudomonas aeruginosa, Bacillus anthracis, and Burkholderia cepacia.

Thus, the methods of the present invention can be used to treat a bacterial infection caused by these species of bacteria. The methods of the present invention can also be used to treat more than one bacterial infection in the respiratory system of the same subject, caused by more than one of these species of bacteria. For example, the methods of the present invention can be used to treat a subject having bacterial infection caused by Staphylococcus aureus (methicillin-resistant or -susceptible) and Pseudomonas aeruginosa; Staphylococcus aureus (methicillin-resistant or -susceptible) and Burkholderia cepacia; Staphylococcus aureus (methicillin-resistant or -susceptible) and Bacillus anthracis; Staphylococcus aureus (methicillin-resistant or -susceptible), Pseudomonas aeruginosa and Burkholderia cepacia; or Staphylococcus aureus (methicillin-resistant or -susceptible), Pseudomonas aeruginosa, Bacillus anthracis and Burkholderia cepacia.

The bacterial infections contemplated herein include both acute and chronic infections. The methods of the present invention can therefore be used to treat bacterial infections where the infection is an acute bacterial infection or a chronic bacterial infection. The present invention is particularly useful for chronic infections, for example the administration can be carried out two, three, four, five, six or seven times a week, or more, for a period of days, weeks, months or years. Particular examples include administration daily for two or four weeks or more; every other day for two or four months or more, etc. For example, administration can be carried out one, two, three, four, five or six times a day for the duration of the infection being treated, with chronic conditions receiving chronic treatments.

### Subject

In each of the embodiments of the present invention, the subject is a human, a non-human primate, horse, cow, goat, sheep, rodent, a companion animal, such as a dog or cat, or other mammal, or an avian species. The subjects to which the methods of the present invention can be applied include subjects having an underlying disease or condition that makes them more susceptible to bacterial infections of the respiratory system. Such subjects include, and are not limited to, those afflicted with cystic fibrosis; lung cancer; an obstructive lung disease, such as chronic obstructive pulmonary disease and asthma; chronic bronchitis; a restrictive lung disease; emphysema; primary and secondary ciliary dyskinesia; sinusitis; mesothelioma; pneumonia; ventilator-associated pneumonia; hospital-acquired pneumonia; community-acquired bacterial pneumonia. Human subjects of both genders and at any stage of development (i.e., neonate, infant, juvenile, adolescent, adult) can be treated according to the present invention. In one aspect of each embodiment, the subject is a human afflicted cystic fibrosis.

### Respiratory System

As used herein, the respiratory system of a subject comprises the airways and lungs of a subject. In particular, the respiratory system comprises: (i) the upper respiratory tract, which includes the nasal passages, paranasal sinuses, and pharynx; (ii) the respiratory airways, which include the larynx, trachea, bronchi, and bronchioles; and (iii) the lungs, which include respiratory bronchioles, alveolar ducts, alveolar sacs, and alveoli.

### Fusidic Acid

Fusidic Acid (FA) has the following structure:

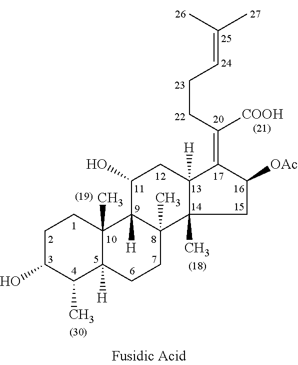

The skilled artisan will understand that for the sake of brevity alone, all references herein to “fusidic acid” or “FA”, alone or in the context of a “pharmaceutical composition” comprising fusidic acid or FA, also refers to the hemihydrate form of the compound, as well as pharmaceutically acceptable salts, other hydrates, solvates, or mixtures thereof, unless otherwise stated.

The term “pharmaceutically acceptable salt” refers to non-toxic base addition salts derived from inorganic and organic bases. Base addition salts include those derived from inorganic bases, such as ammonium or alkali or alkaline earth metal hydroxides, carbonates, bicarbonates, and the like, as well as alkylamine and organic amino salts, such as an ethanolamine salt. Such bases useful in preparing the salts of this invention thus include, and are not limited to, sodium hydroxide, potassium hydroxide, ammonium hydroxide, potassium carbonate, sodium carbonate, sodium bicarbonate, potassium bicarbonate, calcium hydroxide, calcium carbonate, and the like. The potassium and sodium salt forms are exemplified. In particular embodiments, sodium fusidate is a pharmaceutically acceptable salt that is used in the methods of the present invention. Sodium fusidate, also termed CEM-102 herein, has the following structure.

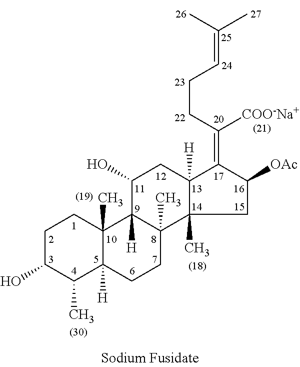

It should be recognized that the particular counter-ion forming a part of any salt of this invention is not of a critical nature, so long as the salt as a whole is pharmacologically acceptable and as long as the counter-ion does not contribute undesired qualities to the salt as a whole.

### Second Antibacterial Agents

Each of the methods of the present invention includes the optional additional step(s) of administering at least one additional antibacterial agent to the subject. Thus the present invention includes methods of treating a bacterial infection in the respiratory system of a subject, comprising (i) administering via inhalation a therapeutically effective amount of a pharmaceutical composition comprising fusidic acid, or a pharmaceutically acceptable salt thereof, and (ii) administering a therapeutically effective amount of at least one additional pharmaceutical composition comprising an antibacterial agent, or a pharmaceutically acceptable salt thereof, to a subject having a bacterial infection of the respiratory system.

In a related embodiment, the present invention also provides methods for delivering fusidic acid and one or more additional antibacterial agents to a subject, comprising (i) administering via inhalation a therapeutically effective amount of a pharmaceutical composition comprising fusidic acid, or a pharmaceutically acceptable salt thereof, to the respiratory system of a subject and (ii) administering a therapeutically effective amount of at least one additional pharmaceutical composition comprising an antibacterial agent, or a pharmaceutically acceptable salt thereof, to the same subject. In certain aspects of this embodiment, the respiratory system of the subject has a bacterial infection.

Such methods that comprise administration of at least one additional pharmaceutical composition comprising an antibacterial agent will typically be practiced by administering only one or two additional antibacterial agents. However, in certain aspects a third or even fourth antibacterial agent can be administered to the subject when the methods of the invention are practiced.

The additional antibacterial agent can be any that has activity against the bacteria that is the basis of the bacterial infection being treated, as well as pharmaceutically acceptable salts, hydrates, solvates, or mixtures thereof, unless otherwise stated. In particular aspects, the additional antibacterial agent is fosfomycin (also known as phosphonomycin or phosphomycin); an aminoglycoside, including streptomycin, neomycin, framycetin, paromomycin, ribostamycin, kanamycin, amikacin, arbekacin, bekanamycin, dibekacin, tobramycin, spectinomycin, hygromycin B, paromomycin, gentamicin, netilmicin, sisomicin, isepamicin, verdamicin and astromicin; a macrolide, including azithromycin, clarithromycin, dirithromycin, erythromycin, roxithromycin, telithromycin and CEM-101 (solithromycin); a glycoprotein, including cinoxacin, flumequine, nalidixic acid, oxolinic acid, piromidic acid, pipemidic acid, rosoxacin, ciprofloxacin, enoxacin, fleroxacin, lomefloxacin, nadifloxacin, norfloxacin, ofloxacin, pefloxacin, rufloxacin, balofloxacin, gatifloxacin, grepafloxacin, levofloxacin, moxifloxacin, pazufloxacin, sparfloxacin, temafloxacin, tosufloxacin, clinafloxacin, gemifloxacin, sitafloxacin, trovafloxacin, prulifloxacin, garenoxacin, and delafloxacin; and an oxazolidinones, including cycloserine, linezolid, torezolid and radezolid.

In each of the methods of the present invention using two or more antibacterial agents, the antibacterial agents may be formulated in a single pharmaceutical composition or in separate pharmaceutical compositions. Thus, the methods can be practiced by administered a single pharmaceutical composition comprising two or more different antibacterial agents to the subject. Alternatively, two or more pharmaceutical compositions, each comprising different antibacterial agents, can be administered to the subject.

Where the pharmaceutical composition comprises a single antibacterial agent, and two or more different pharmaceutical compositions are administered to a subject during treatment, the different pharmaceutical compositions can be administered to the subject sequentially or concurrently. Thus, a pharmaceutical composition comprising an antibacterial agent other than fusidic acid can be administered to a subject prior to, concurrently with, or after administering a pharmaceutical composition comprising fusidic acid.

### Pharmaceutical Compositions

The pharmaceutical compositions of the present invention comprise one or more antibacterial agents and can also comprise one or more of a diluent, carrier and excipient, depending on the identity of the antibacterial agent or agents in the composition. The terms specifically exclude cell culture medium.

Suitable diluents (for both dry and liquid pharmaceutical formulations) are well known to those skilled in the art and include, and are not limited to, saline, buffered saline, dextrose (e.g., 5% dextrose in water), water, glycerol, ethanol, propylene glycol, polysorbate 80 (Tween-80TH), poly(ethylene)glycol 300 and 400 (PEG 300 and 400), PEGylated castor oil (e.g. Cremophor EL), poloxamer 407 and 188, a cyclodextrin or a cyclodextrin derivative.

Carriers are compounds and substances that improve and/or prolong the delivery of an active ingredient to a subject in the context of a pharmaceutical formulation. Carriers may serve to prolong the in vivo activity of a drug or slow the release of the drug in a subject, using controlled-release technologies. Carriers may also decrease drug metabolism in a subject and/or reduce the toxicity of the drug. Carrier can also be used to target the delivery of the drug to particular cells or tissues in a subject. Common carriers (both hydrophilic and hydrophobic carriers) include, and are not limited to, fat emulsions, lipids, PEGylated phospholids, liposomes and lipospheres, microspheres (including those made of biodegradable polymers or albumin), polymer matrices, biocompatible polymers, protein-DNA complexes, protein conjugates, erythrocytes, vesicles and particles.

Excipients included in a pharmaceutical composition have different purposes depending, for example on the nature of the drug, and the mode of administration. Examples of generally used excipients include, without limitation: stabilizing agents, solubilizing agents and surfactants, buffers and preservatives, tonicity agents, bulking agents, lubricating agents (such as talc or silica, and fats, such as vegetable stearin, magnesium stearate or stearic acid), emulsifiers, suspending or viscosity agents, inert diluents, fillers (such as cellulose, dibasic calcium phosphate, vegetable fats and oils, lactose, sucrose, glucose, mannitol, sorbitol, calcium carbonate, and magnesium stearate), disintegrating agents (such as crosslinked polyvinyl pyrrolidone, sodium starch glycolate, cross-linked sodium carboxymethyl cellulose), binding agents (such as starches, gelatin, cellulose, methyl cellulose or modified cellulose such as microcrystalline cellulose, hydroxypropyl cellulose, sugars such as sucrose and lactose, or sugar alcohols such as xylitol, sorbitol or maltitol, polyvinylpyrrolidone and polyethylene glycol), wetting agents, antibacterials, chelating agents, coatings (such as a cellulose film coating, synthetic polymers, shellac, corn protein zein or other polysaccharides, and gelatin), preservatives (including vitamin A, vitamin E, vitamin C, retinyl palmitate, and selenium, cysteine, methionine, citric acid and sodium citrate, and synthetic preservatives, including methyl paraben and propyl paraben), sweeteners, perfuming agents, flavoring agents, coloring agents, administration aids, and combinations thereof. Fusidic acid is acidic and has a bitter taste. Therefore, excipients that mask the acidity and taste of the drug can be included in pharmaceutical compositions comprising fusidic to make the formulation more palatable to a subject.

The pharmaceutical compositions of the present invention are preferably formulated for intranasal or inhalation administration, whether through nasal or buccal administration, or other means that deliver the antibacterial agent(s) to epithelia of the respiratory system, using conventional diluents, carriers, excipients and/or propellants, through formulations such as nose drops, mists, etc. In one embodiment, the pharmaceutical compositions are administered by transbronchoscopic lavage. In particular embodiments, the antibacterial agent(s) are deposited on surfaces of the respiratory system by administering an aerosol suspension of respirable particles comprising of the active agent (i.e., antibacterial agents) through inhalation by the subject. The respirable particles may be liquid or solid (dry).

Aerosols of liquid particles comprising the active agent may be produced by any suitable means, such as with a pressure-driven aerosol nebulizer or an ultrasonic nebulizer. Nebulizers are commercially available devices which transform solutions or suspensions of an active agent into a therapeutic aerosol mist either by means of acceleration of compressed gas, typically air or oxygen, through a narrow vent or orifice, or by means of ultrasonic agitation. Suitable formulations for use in nebulizers consist of the active agent in a liquid carrier. The carrier is typically water (and most preferably sterile, pyrogen-free water) or a dilute aqueous alcoholic solution.

Aerosols of solid particles comprising the active agent may likewise be produced by any solid particulate aerosol generator. Aerosol generators for administering solid particulates to a subject generate a volume of aerosol containing a predetermined metered dose of an active agent at a rate suitable for human administration. One illustrative type of a solid particulate aerosol generator is an insufflator. Suitable formulations for administration by insufflation include fine powders which may be delivered by means of an insufflator or taken into the nasal cavity in the manner of a snuff. In the insufflator, the powder (e.g., a pre-selected dose) is contained in a capsule or cartridge, typically made of gelatin or plastic, that is either pierced or opened in situ and the powder is delivered by air drawn through the device upon inhalation or by means of a manually-operated pump. The powder employed in the insufflator may consist of either the active agent alone, or a powder blend comprising the active agent and a carrier, such as lactose, and an optional surfactant. A second type of illustrative aerosol generator is a metered dose inhaler. Metered dose inhalers are pressurized aerosol dispensers, typically containing a suspension or solution formulation of the active agent in a liquified propellant. These devices discharge the formulation through a valve adapted to deliver a metered volume during use, typically from 10 to 150 ul, to produce a fine particulate spray containing the active agent. Suitable propellants include, and are not limited to, certain chlorofluorocarbon compounds, for example, dichlorodifluoromethane, trichlorofluoromethane, dichlorotetrafluoroethane and mixtures thereof. The formulation may additionally contain one or more co-solvents, for example, ethanol, surfactants, such as oleic acid or sorbitan trioleate, antioxidants and suitable flavoring agents.

Inhalable formulations comprising particles of the antibacterial agents should include particles of respirable size, that is, particles of a size sufficiently small to pass through the mouth and larynx upon inhalation and into the bronchi, bronchioles, and the alveoli of the lungs. In general, particles of less than about 6 microns in size are respirable. In one aspect, the particles of aerosol formations of the present invention are between about 1 and 5 microns. For nasal administration, a particle size in the range of about 10-500 microns is suitable to ensure retention in the nasal cavity. The antibacterial agents themselves may be formulated into particles of the appropriate size, or the agents may be formulated with a carrier of the appropriate size.

The acidity and bitter taste of fusidic acid can also be addressed by preparing nanoparticle formulations of fusidic acid for intranasal or inhalation administration. Nanoparticles formulations generally comprise submicron (<1 μm) colloidal particles, which includes monolithic nanoparticles (nanospheres) in which the drug is adsorbed, dissolved, or dispersed throughout a matrix, and nanocapsules in which the drug is confined to an aqueous or oily core surrounded by a shell-like wall. The drug can alternatively be covalently attached to the surface or into the matrix. Nanoparticles can be made from biocompatible and biodegradable materials such as polymers, either natural (e.g., gelatin, albumin) or synthetic (e.g., polylactides, polyalkylcyanoacrylates), or solid lipids. In the body, the drug loaded in nanoparticles is typically released from the matrix by diffusion, swelling, erosion, or degradation. Thus, the formulations of the present invention may contain microspheres, microcapsules, nanoparticles or the like.

The pharmaceutical compositions of the present invention may also be formulated for parenteral, oral or intraocular administration. Parenteral modes of administration include intramuscular (IM) and intravenous (IV). Any known device useful for parenteral injection or infusion of drug formulations can be used to effect such administration.

Formulations for parenteral administration can be in the form of aqueous or non-aqueous isotonic sterile injection solutions, suspensions or fat emulsions. The parenteral form used for injection must be fluid to the extent that easy syringability exists. These solutions or suspensions can be prepared from sterile concentrated liquids, powders or granules.

Excipients used in parenteral preparations also include, without limitation, stabilizing agents (e.g. carbohydrates, amino acids and polysorbates, such as 5% dextrose), solubilizing agents (e.g. cetrimide, sodium docusate, glyceryl monooleate, polyvinylpyrolidone (PVP) and polyethylene glycol (PEG)), surfactants (e.g. polysorbates, tocopherol PEG succinate, poloxamer and Cremophor™), buffers (e.g. acetates, citrates, phosphates, tartrates, lactates, succinates, amino acids and the like), antioxidants and preservatives (e.g. BHA, BHT, gentisic acids, vitamin E, ascorbic acid, sodium ascorbate and sulfur containing agents such as sulfites, bisulfites, metabisulfites, thioglycerols, thioglycolates and the like), tonicity agents (for adjusting physiological compatibility), suspending or viscosity agents, antibacterials (e.g. thimersol, benzethonium chloride, benzalkonium chloride, phenol, cresol and chlorobutanol), chelating agents, and administration aids (e.g. local anesthetics, anti-inflammatory agents, anti-clotting agents, vaso-constrictors for prolongation and agents that increase tissue permeability), and combinations thereof.

The parenteral unit dosage form can be a ready-to-use solution of the antibacterial agent in a suitable carrier in sterile, hermetically sealed ampoules or in sterile pre-loaded syringes. The suitable carrier optionally comprises any of the above-mentioned excipients. Alternatively, the unit dosage can be in a concentrated liquid, powder or granular form for ex tempore reconstitution in the appropriate pharmaceutically acceptable carrier, such as sterile water, at the time of delivery. In addition to the above-mentioned excipients, powder forms optionally include bulking agents (e.g. mannitol, glycine, lactose, sucrose, trehalose, dextran, hydroxyethyl starch, ficoll and gelatin), and cryo or lyoprotectants.

In intramuscular preparations, a sterile formulation of the pharmaceutical compositions of the present invention can be dissolved and administered in a pharmaceutical diluent such as Water-for-Injection (WFI), physiological saline or 5% dextrose in water. A suitable insoluble form of the pharmaceutical compositions may be prepared and administered as a suspension in an aqueous base or a pharmaceutically acceptable oil base, e.g. an ester of a long chain fatty acid such as ethyl oleate.

In intravenous (IV) use, a sterile formulation of the pharmaceutical compositions of the present invention and optionally one or more additives, including solubilizers or surfactants, can be dissolved or suspended in any of the commonly used intravenous fluids and administered by infusion. Intravenous fluids include, without limitation, physiological saline, phosphate buffered saline, 5% dextrose in water, 0.002% polysorbate 80 (Tween-80TH) in water or Ringer's™ solution.

For oral use, the oral pharmaceutical composition may be made in the form of a unit dosage containing a therapeutically effective amount of the pharmaceutical compositions. Solid formulations such as tablets and capsules are particularly useful. Sustained released or enterically coated preparations may also be devised. For pediatric and geriatric applications, suspension, syrups and chewable tablets are especially suitable. For oral administration, the pharmaceutical compositions are in the form of, for example, tablets, capsules, suspensions or liquid syrups or elixirs, wafers and the like. For general oral administration, excipient or additives include, but are not limited to inert diluents, fillers, disintegrating agents, binding agents, wetting agents, lubricating agents, sweetening agents, flavoring agents, coloring agents and preservatives.

For therapeutic purposes, the tablets and capsules can contain, in addition to the antibacterial agent, conventional carriers such as: inert diluents (e.g., sodium and calcium carbonate, sodium and calcium phosphate, and lactose), binding agents (e.g., acacia gum, starch, gelatin, sucrose, polyvinylpyrrolidone (Povidone), sorbitol, tragacanth methylcellulose, sodium carboxymethylcellulose, hydroxypropyl methylcellulose, and ethylcellulose), fillers (e.g., calcium phosphate, glycine, lactose, maize-starch, sorbitol, or sucrose), wetting agents, lubricating agents (e.g., metallic stearates, stearic acid, polyethylene glycol, waxes, oils, silica and colloical silica, silicon fluid or talc), disintegrating agents (e.g., potato starch, corn starch and alginic acid), flavouring (e.g. peppermint, oil of wintergreen, fruit flavoring, cherry, grape, bubblegum, and the like), and coloring agents. Carriers may also include coating excipients such as glyceryl monostearate or glyceryl distearate, to delay absorption in the gastrointestinal tract.

Oral liquid preparations, generally in the form of aqueous or oily solutions, suspensions, emulsions or elixirs, may contain conventional additives such as suspending agents, emulsifying agents, non-aqueous agents, preservatives, coloring agents and flavoring agents. Examples of additives for liquid preparations include, and are not limited to, acacia, almond oil, ethyl alcohol, fractionated coconut oil, gelatin, glucose syrup, glycerin, hydrogenated edible fats, lecithin, methyl cellulose, microcrystalline cellulose, methyl or propyl para-hydroxybenzoate, propylene glycol, sorbitol, or sorbic acid.

The therapeutically effective amount of any of the pharmaceutical compositions, and the amounts sufficient to achieve the stated goals of the methods disclosed herein, will vary depending upon the physical characteristics of the subject, the age of the subject, the severity of the subject's symptoms, the identity of the bacteria, the location of the bacterial infection(s), the formulation and the means used to administer the antibacterial agent(s), the number of doses being administered to the subject over the course of treatment, and the method being practiced. The specific doses for a given subject are usually set by the judgment of the attending physician. However, general ranges and some non-limiting specific examples are provided in the following paragraphs.

### Fusidic Acid Dosage Forms

The pharmaceutical formulations comprising fusidic acid or salts thereof of the present invention include aerosol formulations and lavage solutions. Aerosol formulations comprising fusidic acid for use in the methods of the present invention can comprise fusidic acid formulated in an aqueous solution (comprising, for example, nitrogen, sodium chloride, sodium hydroxide, sterile water for injection and sulfuric acid) or a dry powder for administration, for example, by a nebulizer. Solutions of fusidic acid for administration via bronchoalveolar lavage can comprise a variety of different aqueous carriers including, but not limited to, 0.9% saline, buffered saline, physiologically compatible buffers and the like, in addition to the drug. Such formulations and solutions comprising fusidic acid can comprise between about 50 mg and about 1500 mg of fusidic acid. Additional ranges include between about 200 mg and about 1500 mg, between about 100 mg and about 1400 mg, between about 200 mg and about 1000 mg, between about 250 mg and about 750 mg, between about 400 mg and about 800 mg, and between about 300 mg and about 600 mg. In particular aspects, the formulations and solutions can comprise about 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1075, 1100, 1125, 1150, 1175, 1200, 1225, 1250, 1275, 1300, 1325, 1350, 1375, 1400, 1425, 1450, 1475, 1500 mg or more fusidic acid. The aerosol formulations are typically in a volume of between about 1 ml and 10 ml. In particular aspects, the volume is about 5 ml or less, 4.5 ml or less, 4 ml or less, about 3.75 ml or less, about 3.5 ml or less, about 3.25 ml or less, or about 3.0 ml or less. Pharmaceutical compositions comprising fusidic acid can be administered 1, 2, 3, 4 or more times per day. Inhalation administration can extend over a period of about 5, 6, 7, 8, 9, 10, 15, 20, 25, 30 or more minutes via a nebulizer. In particular aspects, the period of inhalation administration can be about 10 minutes or less, about 8 minutes or less, or about 6 minutes or less. In a particular formulation for inhalation administration, the pharmaceutical composition comprises about 250, 300, 350, 400, 450, 500, 550, 600, 650, 700, 750 or 800 mg of fusidic acid in a volume of aqueous solution of about 4 ml.

### Tobramycin Dosage Forms

The pharmaceutical formulations comprising tobramycin or salts thereof of the present invention include aerosol formulations. Aerosol formulations comprising tobramycin for use in the methods of the present invention are well known in the art (see, e.g., U.S. Pat. Nos. 5,508,269; 6,987,094) and they can comprise, for example, tobramycin sulfate formulated in an aqueous solution (comprising, for example, nitrogen, sodium chloride, sodium hydroxide, sterile water for injection and sulfuric acid), preferably for administration by a nebulizer. Aerosol formulations comprising tobramycin can comprise between about 50 mg and about 600 mg of tobramycin sulfate, for example between about 100 mg and about 500 mg, or between about 200 mg and about 400 mg. In particular aspects, the aerosol formulations comprise about 100, 150, 200, 250, 300, 350, 400 mg or more tobramycin sulfate. The aerosol formulations are typically in a volume of between about 1 ml and 10 ml, for example, the volume is about 5 ml or less, 4.5 ml or less, 4 ml or less, about 3.75 ml or less, about 3.5 ml or less, about 3.25 ml or less, or about 3.0 ml or less. Pharmaceutical compositions comprising tobramycin for inhalation administration can be administered 1, 2, 3, 4 or more times per day, over a period of about 5, 6, 7, 8, 9, 10, 15, 20, 25, 30 or more minutes via a nebulizer. In particular aspects, the period of administration is about 10 minutes or less, about 8 minutes or less, or about 6 minutes or less. In particular aerosol formulations, tobramycin sulfate is formulated in single-use 5 mL ampules containing about 300 mg tobramycin and about 11.25 mg sodium chloride in sterile water. Sulfuric acid and sodium hydroxide are added to adjust the pH to 6.0. The formulation is administered BID using an alternating 28 day on/off period (i.e., the formulation is administered for 28 days, followed by 28 days without treatment, and then returning to treatment for 28 days, etc).

The pharmaceutical formulations comprising tobramycin or salts thereof of the present invention include intravenous (IV) formulations. IV formulations comprising tobramycin are also well known in the art and they can be administered, for example, to a subject in a dosage of between about 0.1 to 10 mg/kg/day, for example about 1, 2, 3, 4, 5, 6, 7, 8, 9, 10 or more mg/kg, by IV infusion over approximately 10, 20, 30, 40 50, 60, 70, 80, 90 or more minutes, every 4, 6, 8, 10, 12, 14 or more hours. In these formulations, tobramycin sulfate can be reconstituted in sterile water for injection (WFI), and then diluted in 5% dextrose in water or 0.9% sodium chloride to a total volume of between about 50 to 100 mL. In a particular embodiment, an IV formulation comprising tobramycin can be administered to an adult human in a dosage of about 3-5 mg/kg/day in 3-4 equal divided doses every 6-8 hours. For pediatric subjects, between about 6-7.5 mg/kg/day tobramycin in 3-4 equal divided doses can be administered. For patients with cystic fibrosis, an initial dosage regimen of 10 mg/kg/day in 4 equally divided doses is recommended. In particular aspects, methods of treatment using tobramycin administered via IV or IM are discontinued after a maximum of 10 days due to the potential for neurotoxicity. Appropriate dosing for a pharmaceutical composition comprising tobramycin administered via intramuscular injection is the same as for IV administration.

### Amikacin Dosage Forms

The pharmaceutical formulations comprising amikacin or salts thereof of the present invention include aerosol formulations. Aerosol formulations comprising amikacin for use in the methods of the present invention are well known in the art (see, e.g., U.S. Pat. Nos. 7,718,189; 5,508,269; U.S. Appln. Publication No. 20090104256) and they can comprise, for example, amikacin sulfate formulated in an aqueous solution, preferably for administration by a nebulizer. Aerosol formulations comprising amikacin can comprise between about 50 mg and about 800 mg amikacin sulfate, for example between about 200 mg and about 600 mg, or between about 300 mg and about 500 mg. In particular aspects, the aerosol formulations comprise about 300, 350, 400, 450, 500, 550, 600 mg or more amikacin sulfate. The aerosol formulations are typically in a volume of between about 1 ml and 10 ml. In particular aspects, the volume is about 4 or less, about 3.75 ml or less, about 3.5 ml or less, about 3.25 ml or less, or about 3.0 ml or less. Pharmaceutical compositions comprising amikacin for inhalation administration can be administered 1, 2, 3, 4 or more times per day, over a period of about 5, 6, 7, 8, 9, 10, 15, 20, 25, or more minutes via a nebulizer. In particular aspects the period of administration is about 10 minutes or less, about 8 minutes or less, or about 6 minutes or less. In a particular formulation, amikacin sulfate can be formulated in dosage units of about 400 mg in about 4 ml of aqueous solution.

The pharmaceutical formulations comprising amikacin or salts thereof of the present invention include IV and intramuscular (IM) formulations. IV and IM formulations comprising amikacin are also well known in the art (see, e.g., U.S. Pat. No. 3,781,268) and they can be administered, for example, in a dosage of between about 0.1 to 30 mg/kg/day, for example, about 4, 5, 6, 7, 8, 9, 10 or more mg/kg, over approximately 10, 20, 30, 40, 50, 60 or more minutes for IV infusion, every 8, 10, 12, 14, 16 or more hours. Amikacin is generally supplied in vials comprising 100 or 500 mg amikacin sulfate in 2 ml sterile water for injection, sodium metabisulfite, and sodium citrate dehydrate, adjusted to a pH of 4.5 with sulfuric acid. In a particular embodiment, an IV formulation comprising amikacin can be administered to an adult human in a dosage of about 6-8 mg/kg/day in 2-3 equal divided doses every 8-12 hours. The solution for intravenous use can be prepared by adding the contents of a 500 mg vial to 100 or 200 mL of sterile diluent such as 0.9% sodium chloride injection or 5% dextrose injection. Appropriate dosing for IM injection is similar as for IV administration. In particular, adults, children and older infants can be administered 15 mg/kg/day divided into 2 or 3 equal doses administered at equally-divided intervals, i.e., 7.5 mg/kg ql2 h or 5 mg/kg q8 h.

### Fosfomycin Dosage Forms

The pharmaceutical formulations comprising fosfomycin or salts thereof of the present invention include oral formulations and formulations for pulmonary delivery. Oral formulations comprising fosfomycin for use in the methods of the present invention are well known in the art and they can comprise fosfomycin tromethamine dissolved in water. Fosfomycin is typically supplied in a sachet containing dry fosfomycin tromethamine powder and the following inactive ingredients: mandarin flavor, orange flavor, saccharin, and sucrose. The contents of the sachet are mixed with water and then drunk by a subject. Oral formulations of fosfomycin can contain between about 0.5 and 10 g of fosfomycin, for example about 1, 2, 3, 4, 5, 6, 7, 8, 9 or 10 g of fosfomycin. Pharmaceutical compositions comprising fosfomycin can be administered to a subject 1, 2, 3 or more times per day. In a particular embodiment, 3 g of fosfomycin are dissolved in 3-4 ounces of water and drunk by a subject once a day.

The pharmaceutical formulations comprising fosfomycin or salts thereof of the present invention also include aerosol formulations. Aerosol formulations comprising fosfomycin for use in the methods of the present invention are well known in the art (see, e.g., U.S. Patent Appln. Publ. No. 20100063005). Aerosol formulations of fosfomycin are delivered to a subject in doses of between about 1 and 100 mg/kg, an include the ranges of about 5 mg to 90 mg, about 10 mg to 80 mg, about 15 mg to 80 mg, and about 25 mg to 75 mg per kilo body weight, administered 1, 2, 3, 4, 5, 6, or 7 times daily.

### Levofloxacin Dosage Forms

The pharmaceutical formulations comprising levofloxacin or salts thereof of the present invention include formulations for oral administration. Such formulations for use in the methods of the present invention are well known in the art and they include levofloxacin in the form of a tablet. The tablet form of levofloxacin is generally prescribed in a dosage of between about 100 and 1000 mg, for example, about 250, 300, 350, 400, 450, 500, 550, 600, 650 700 or 750 mg, and administered 1, 2, 3 or more times daily. In a particular embodiment, 500 mg or 750 mg levofloxacin is administered to a subject once a day.

The pharmaceutical formulations comprising levofloxacin or salts thereof of the present invention also include IV infusion formulations. IV infusion formulations comprising levofloxacin are also well known in the art and they can be administered, for example, in a dosage of between about 100 and 1000 mg, for example, about 250, 300, 350, 400, 450, 500, 550, 600, 650 700 or 750 mg, over approximately 30, 40, 50, 60, 70, 80, 90, 100, 110 or 120 minutes, or more, every 12, 18, 24, 36, 42 or 48 hours, or more. Levofloxacin is generally supplied in premixed, single-use containers comprising 250, 500 or 750 mg levofloxacin in 5% dextrose, at a concentration of 5 mg/ml. Where levofloxacin is supplied in a higher concentration, lower volume container, it may be diluted to about 5 mg/ml in an appropriate buffered solution for IV infusion to a subject. Levofloxacin is generally infused intravenously slowly over a period of 60 or 90 minutes, depending on the dosage.

The pharmaceutical formulations comprising levofloxacin or salts thereof of the present invention further includes aerosol formulations. Aerosol formulations comprising levofloxacin for use in the methods of the present invention are well known in the art (see, e.g., U.S. Patent Appln. Publ. Nos. 20100158957; 20100037890; 20100087416). Aerosol formulations comprising levofloxacin can comprise between about 50 mg and about 800 mg levofloxacin, for example between about 200 mg and about 600 mg, or between about 300 mg and about 500 mg. In particular aspects, the aerosol formulations comprise about 100, 150, 200, 250, 300, 350 or 400 mg, or more, levofloxacin. Pharmaceutical compositions comprising levofloxacin for inhalation administration can be administered 1, 2, 3, 4 or more times per day, over a period of about 5, 6, 7, 8, 9, 10, 15, 20, 25, 30 or more minutes via a nebulizer.

### Inhalation Devices

Suitable inhalation devices for use with the pharmaceutical compositions and methods of the present invention are readily available and known to the skilled artisan. In order to deliver a relatively small volume of relatively highly concentrated pharmaceutical compositions comprising antibacterial agents to a subject via inhalation in the relatively short period of time, pharmaceutical compositions are preferably administered using an inhalation device having a relatively high rate of aerosol output. The rate of aerosol output by the inhalation devices that can be used in conjunction with the methods of the present invention is at least about 2, 3, 4, 5, 6, 7, 8, 9, or 10 ul/sec, preferably at least about 3 ul/sec, more preferably at least about 4 ul/sec. Useful devices should exhibit high emitted-dose efficiency (i.e., low residual volume in the device), releasing at least about 55% of the nominal dose as an aerosol, at least about 75%, at least about 80%, or at least about 85% of the loaded dose as aerosol for inhalation by the patient. While inhalation devices that can be used in conjunction with the methods of the present invention can continually release aerosolized drug throughout the delivery period, without regard to whether the patient is inhaling, exhaling or in a static portion of the breathing cycle, inhalation devices use with the methods are preferably breath actuated, thereby restricted to delivery of drug to the subject during actual inhalation by the subject. Representative inhalation devices suitable for use in conjunction with the methods of the present invention include an air-jet nebulizer coupled with a compressor capable of higher than conventional output pressures, such as the PARI LC PLUS™ jet nebulizer (PARI GmbH, Stamberg, Germany) driven by a Invacare MOBILAIRE™ compressor (Invacare Corporation, Elyria, Ohio), and the Aerodose™ inhaler (Aerogen, Inc., Sunnyvale, Calif.). The pharmaceutical formulations may also be delivered by via (a) facemasks, or (b) via endotracheal tubes in intubated patients during mechanical ventilation. As suggested above, the pharmaceutical formulations may also be delivered to the respiratory system of a subject via a lavage fluid administered via a bronchoscope as a bronchoalveloar lavage or as a blind intratracheal wash or lavage.

### Bronchodilator

Because the administration of agents to the respiratory system of some subjects can induce bronchoconstriction in the subject, each of the methods of the present invention can include the administration of a bronchodilator to said subject prior to or concurrently with a pharmaceutical composition comprising one of the antibacterial agents of the present invention in an amount sufficient to inhibit bronchoconstriction. Suitable bronchodilators will depend on the identity of the antibacterial agent being administered to a subject, but can include, and are not limited to, beta-adrenergic agonists, including but not limited to: epinephrine, isoproterenol, fenoterol, albuterol, terbutaline, pirbuterol, bitolterol, metaproterenol, isoetharine, salmeterol and xinafoate, as well as anticholinergic agents including but not limited to: ipratropium bromide, as well as compounds such as theophylline and aminophylline.

The present invention also encompasses kits comprising the pharmaceutical compositions of the present invention, means for administration (e.g., an inhalation apparatus) and instructions regarding administration. For example, a kit can contain single-use ampules containing pre-measured dosages of fusidic acid and a suitable carrier, along with instructions for using the ampules in a nebulizer. As another example, the kit can contain pressurized delivery devices containing pre-measured dosages of fusidic acid and a suitable carrier, along with instructions for use. Equipment used for administering the pharmaceutical compositions is well known in the art and they are described in detail, such in Remington: The Science and Practice of Pharmacy, 19^{th} Edition, 1995, Mac Publishing Company, Easton, Pa., pages 1676-1692.

As used herein, the terms “dose”, “dosage”, “unit dose”, “unit dosage”, “effective dose” and related terms refer to physically discrete units that contain a predetermined quantity of active ingredient calculated to produce a desired therapeutic effect. These terms are synonymous with the therapeutically effective amounts and amounts sufficient to achieve the stated goals of the methods disclosed herein.

As used herein, the terms “treat”, “treating” and “treatment” have their ordinary and customary meanings, and include one or more of, ameliorating a symptom of a bacterial infection in a subject, blocking or ameliorating a recurrence of a symptom of a bacterial infection in a subject, decreasing in severity and/or frequency a symptom of a bacterial infection in a subject, stasis, decreasing, or inhibiting growth of bacteria causing a bacterial infection in a subject, and killing bacteria causing a bacterial infection in a subject. Treatment means ameliorating, blocking, reducing, decreasing or inhibiting by about 1% to about 100% versus a subject to which a pharmaceutical composition has not been administered. The ameliorating, blocking, reducing, decreasing or inhibiting can be about 100%, 99%, 98%, 97%, 96%, 95%, 90%, 80%, 70%, 60%, 50%, 40%, 30%, 20%, 10%, 5% or 1% versus a subject to which a pharmaceutical composition has not been administered. The term “therapeutically effective amount” is an amount of the active agent or agents in a pharmaceutical composition that is sufficient to treat a subject having a bacterial infection.

### EXAMPLES

### Example 1

### Activity of CEM-102, Alone and in Combination with Tobramycin and Amikacin, Against P. Aeruginosa, MRSA, and B. Cepacia

This study tested activity of CEM-102 (fusidic acid) against Pseudomonas aeruginosa, methicillin-resistant Staphylococcus aureus (MRSA) and Burkholderia cepacia strains, alone and in combination with amikacin or tobramycin.

### Materials and Methods

Strains. Two strains each of mucoid Pseudomonas aeruginosa (both pyocyanin positive) and 40 MRSA (only one strain with gold colonies), isolated within the past 12 months and beyond from patients at in cystic fibrosis clinic, were tested. Additionally, two B. cepacia strains were acquired from Hershey Medical Center. All strains were identified by standard methods. Only one strain per patient was tested. MLVA was done on all strains, to examine clonality and to ensure that testing was not being limited to only one or a few clones. Strains were stored in skim milk at −70° C. until use.

Susceptibility testing. Original MICs of each strain to CEM-102 and other comparators was tested by CLSI microdilution methodology. Trays were obtained from Trek, Inc., Cleveland, Ohio. Time-kill macrobroth MIC dilution by CLSI was performed for all synergy testing.

Synergy testing. Two of the MRSA strains were chosen and tested for synergy, together with the four Gram-negative strains mentioned above. Broth macrodilution formed the basis of MICs used in time-kill experiments, as detailed below. The kill kinetics of each drug was tested alone by incubating an initial inoculum of 5×10⁵ to 5×10⁶ cfu/ml with drug concentrations at the MIC, three dilutions above and three dilutions below the MIC (1/2, 1/4 and 1/8×MIC). Viability counts were performed after 0, 3, 6, 12 and 24 h incubation at 37° C. in a shaking water bath by plating onto trypticase soy-5% sheep blood agar plates.

After initial time-kills with drugs alone had been done, CEM-102 was combined with amikacin or tobramycin. Combinations were tested 1-2 dilutions below the MIC (1/2×MIC and 1/4×MIC) of each drug. Inocula and time-kill methodology were as above when the drugs alone are tested. Concentrations in synergy time-kill tests were selected such that one of the two drugs yields a growth curve similar to that of the drug-free control, while the other drug was more active.

MICs were assayed by standard methodology. Synergy was defined as a ≧2 log₁₀ decrease in cfu/ml between the combination and its most active constituent after 3, 6, 12 and 24 h, with the number of surviving organisms in the presence of the combination ≧2 log₁₀ cfu/ml below the starting inoculum. At least one of the drugs in the combination was present in a concentration which did not significantly affect the growth curve of the organism when used alone. Antagonism was defined as a ≧2 log₁₀ increase in cfu/ml between the combination and its most active constituent after 3, 6, 12 and 24 h, with the number of surviving organisms in the presence of the combination ≧2 log₁₀ cfu/ml above the starting inoculum.

### Results

Each individual strain tested proved to be an individual clone. Compiled MIC (pg/ml) data from S. aureus (MRSA) is provided in Table 1.

TABLE 1
Microdilution MICs (μg/ml) of all compounds against
40 MRSA strains from cystic fibrosis patients.

| Drug | Range | MIC₅₀ | MIC₉₀ |
|---|---|---|---|
| CEM-102 | 0.12-0.5 | 0.12 | 0.25 |
| Vancomycin | 0.5-1 | 0.5 | 1 |
| Teicoplanin | 0.25-1 | 0.5 | 1 |
| Daptomycin | 0.5-1 | 0.5 | 1 |
| Tigecycline | 0.12-0.25 | 0.12 | 0.25 |
| Azithromycin | 1-≧32 | ≧32 | ≧32 |
| Clarithromycin | 0.25-≧32 | ≧32 | ≧32 |
| Linezolid | 1-4 | 2 | 2 |
| Quinupristin/ | 0.25-1 | 0.5 | 1 |
| dalfopristin | | | |

As can be seen, CEM-102 was potent at MICs between 0.125 and 0.5 against all strains tested. Vancomycin and teicoplanin were also active at MICs 0.25-1, linezolid at MICs 1-4 and quinupristin/dalfopristin at MICs 0.25-1. Most strains (38 of 40) were resistant (>32) to azithromycin and clarithromycin. Microbroth MICs for the 4 Gram-negative rods are presented in Tables 2 and 3, and time-kill macrobroth MIC data can be found in Table 4.

TABLE 2
Macrobroth Dilution MICs (μg/ml) of all compounds against
two P. aeruginosa strains from cystic fibrosis patients.

| Drug | Range |
|---|---|
| CEM-102 | >64 |
| Amikacin | 2-8 |
| Tobramycin | 0.25-1.0 |

TABLE 3
Macrobroth Dilution MICs (μg/ml) of all compounds against
two B. cepacia strains from cystic fibrosis patients.

| Drug | Range |
|---|---|
| CEM-102 | >64 |
| Amikacin | 256 |
| Tobramycin | 128 |

TABLE 4
Time-kill Macrobroth MICs (μg/ml) of all compounds
against 6 strains from cystic fibrosis patients.

| Strain | CEM-102 | Tobramycin | Amikacin |
|---|---|---|---|
| SA 2230 | 0.5 | 4.0 | 32.0 |
| SA 2232 | 0.25 | NT^{a} | 64.0 |
| PSAR 461 | NT | 2.0 | 8.0 |
| PSAR 468 | 256 | 1.0 | 4.0 |
| BCEP 953 | 512 | 128 | 512 |
| BCEP 954 | 512 | 128 | 256 |

^{a}NT; not tested

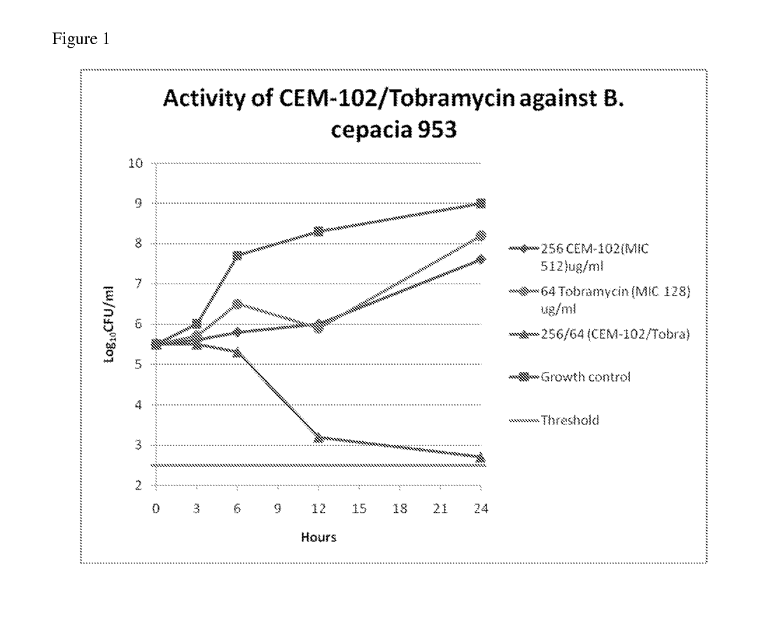

Synergy time-kill data (Tables 5 and 6) were as follows. With CEM-102/tobramycin, synergy was found at (0.125/1) concentration at 24 h for one MRSA strain. All other time points and combinations were indifferent for the two MRSA strains. One strain of MRSA was not tested with tobramycin in combination because of its very high MIC (>512 ug/ml). All time points and combinations were indifferent with the two P. aeruginosa strains. One P. aeruginosa strain was not tested with CEM-102 in combination (MIC>512 ug/ml). One B. cepacia strain showed synergy at 12 and 24 h with CEM-102/tobramycin at 256/64 and 256/32 μg/ml, respectively (FIG. 1). The two B. cepacia strains both showed synergy with the CEM-102/amikican combination at 128/128 μg/ml. All other time points and combinations were indifferent with the two B. cepacia strains.

TABLE 5
Results of In Vitro Antimicrobial Combinations
with CEM102 Studied by Time-kill

| | CEM-102^{d}/Tobramycin^{c} | | | | CEM-102^{d}/Amikacin | | | |
|---|---|---|---|---|---|---|---|---|
| | 3 h^{a} | 6 h^{a} | 12 h^{a} | 24 h^{a} | 3 h | 6 h | 12 h | 24 h |
| Synergy | 0^{b} | 0 | 1 | 2 | 0 | 0 | 0 | 3 |
| Indifference | 4 | 4 | 3 | 2 | 5 | 5 | 5 | 2 |
| Antagonism | 0 | 0 | 0 | 0 | 0 | 0 | 0 | 0 |

^{a}time-point (hours)
^{b}number of strains (strains tested)
^{c}one strain (MRSA 2232) not tested (MIC >512 ug/ml)
^{d}one strain (PSAR 461) not tested (MIC >512 ug/ml)

TABLE 6
Results of In Vitro Antimicrobial Combinations
with CEM102 Studied by Time-kill

| | CEM-102/Tobramycin | | | | CEM-102Amikacin | | | |
|---|---|---|---|---|---|---|---|---|
| | 3 h^{a} | 6 h^{a} | 12 h^{a} | 24 h^{a} | 3 h | 6 h | 12 h | 24 h |
| SA2230 | IND | IND | IND | SYN^{b} | IND | IND | IND | IND |
| | | | | (.125/1) | | | | |
| SA2232 | NT^{c} | NT | NT | NT | IND | IND | IND | IND |
| PSAR461 | NT | NT | NT | NT | NT | NT | NT | NT |
| PSAR468 | IND | IND | IND | IND | IND | IND | IND | IND |
| BCEP953 | IND | IND | SYN | SYN | IND | IND | IND | SYN |
| | | | (256/64) | (256/32) | | | | (128/128) |
| BCEP954 | IND | IND | IND | IND | IND | IND | IND | SYN |
| | | | | | | | | (128/128) |

^{a}time-point (hours)
^{b}IND—indifference; SYN—synergy; ANT—antagonism
^{c}NT; not tested (MIC >512 ug/ml)

No correlation between pigment and any MRSA results were found. When both mucoid P. aeruginosa strains were subcultured for a few days, viscosity disappeared but reappeared when they were re-exposed to all combinations.

CEM-102 was very potent against all strains of MRSA tested. For MRSA, clinically achievable synergy was observed with strain SA 2230, with CEM-102 combined with tobramycin.

All documents, books, manuals, papers, patents, published patent applications, guides, abstracts and other reference materials cited herein are incorporated by reference in their entirety. While the foregoing specification teaches the principles of the present invention, with examples provided for the purpose of illustration, it will be appreciated by one skilled in the art from reading this disclosure that various changes in form and detail can be made without departing from the true scope of the invention.

1-22. (canceled)

23. A method of treating a bacterial infection in the respiratory system of a subject, comprising administering via inhalation a therapeutically effective amount of a pharmaceutical composition comprising fusidic acid, or a pharmaceutically acceptable salt thereof, to the respiratory system of a subject having a bacterial infection therein.

24. (canceled)

25. The method of claim 23, wherein the bacterial infection is an infection caused by one or more bacterial species selected from the group consisting of Staphylococcus aureus (methicillin-resistant or -susceptible), Pseudomonas aeruginosa, Bacillus anthracis, and Burkholderia cepacia.

26. The method of claim 23, wherein the therapeutically effective amount of the pharmaceutical composition comprises between about 200 mg and about 1500 mg of fusidic acid, or a pharmaceutically acceptable salt thereof.

27. The method of claim 23, wherein the therapeutically effective amount of the pharmaceutical composition comprises between about 400 mg and about 800 mg of fusidic acid, or a pharmaceutically acceptable salt thereof.

28. The method of claim 23, wherein the pharmaceutical composition is administered to the subject once, twice or thrice daily.

29-34. (canceled)

35. A method of treating a bacterial infection in the respiratory system of a subject, comprising administering via inhalation a dose of about 4.0 ml or less of a nebulized aerosol formulation comprising from about 200 mg to about 1500 mg of fusidic acid, or a pharmaceutically acceptable salt thereof, in a time period of about 10 minutes or less to a subject having a bacterial infection of the respiratory system.

36. (canceled)

37. The method of claim 35, wherein said administering is via an inhalation device having a rate of aerosol output of not less than about 4 ul/sec, that releases about 75% of the loaded dose, and that produces aerosol particles having particle sizes between about 1 micron and about 5 micron.

38. The method of claim 35, wherein the dose is about 3.75 ml or less of the nebulized aerosol formulation.

39. The method of claim 35, wherein the nebulized aerosol formulation comprises from about 400 mg to about 800 mg of fusidic acid, or a pharmaceutically acceptable salt thereof.

40. The method of claim 35, wherein the nebulized aerosol formulation comprises from about 300 mg to about 600 mg of fusidic acid, or a pharmaceutically acceptable salt thereof.

41. The method of claim 35, wherein the bacterial infection is an infection caused by one or more bacterial species selected from the group consisting of Staphylococcus aureus (methicillin-resistant or -susceptible), Pseudomonas aeruginosa, Bacillus anthracis, and Burkholderia cepacia.

42. The method of claim 23 or 35, wherein the bacterial infection is a chronic bacterial infection.

43. The method of claim 23 or 35, wherein the subject is a human.

44. The method of claim 23 or 35, further comprising administering a therapeutically effective amount of a second pharmaceutical composition to said subject prior to, concurrently with, or after administering the fusidic acid, or pharmaceutically acceptable salt thereof, wherein the second pharmaceutical composition comprises tobramycin, amikacin, fosfomycin, levofloxacin, or a pharmaceutically acceptable salt thereof.

45. The method of claim 44, wherein the second pharmaceutical composition is administered to the respiratory system of the subject by inhalation.

46. The method of claim 44, wherein the second pharmaceutical composition is administered to the subject intravenously or intramuscularly.

47-55. (canceled)

56. The method of claim 23 or 35, further comprising administering a bronchodilator to said subject prior to or concurrently with the fusidic acid, or pharmaceutically acceptable salt thereof, in an amount sufficient to inhibit bronchoconstriction.

57-60. (canceled)

61. The method of claim 23 or 35, wherein the subject has cystic fibrosis.
